(19) United States
(12) Patent Application Publication
(10) Pub. No.: US 2015/0196704 A1
(43) Pub. Date: Jul. 16, 2015

(54) IMPLANTABLE HIGH FLOW MULTI-WINDOW VASCULAR ACCESS PORT CATHETER

(71) Applicant: Grit Armstrong Adler, Thonotosassa, FL (US)

(72) Inventor: Grit Armstrong Adler, Thonotosassa, FL (US)

(21) Appl. No.: 14/340,380

(22) Filed: Jul. 24, 2014

Related U.S. Application Data
(63) Continuation-in-part of application No. 14/156,948, filed on Jan. 16, 2014.

(51) Int. Cl. A61M 1/36; A61M 39/10

(52) U.S. Cl. CPC A61M 1/3653; A61M 39/10

(57) ABSTRACT

A subcutaneous implantable vascular access port reduces the risk of infection by creating a larger surface area for needle access with multiple palpable windows. These multiple needle access sites inhibit skin breakdown and thus reducing the risk of infection. This dual lumen port catheter is designed for both high pressure and high flow fluid volume blood flow rates. The port is suitable for dialysis, electrophoresis and a variety of other medical treatment functions. The interchangeable replaceable parts of the port assembly allow easier maintenance and more precise placement positions within the patient.

### CROSS-REFERENCE TO RELATED APPLICATIONS

This application is a continuation-in-part of U.S. patent application Ser. No. 14/156,948 (Attorney Docket No. 46919-703.201), filed Jan. 16, 2014, the entire content of which is incorporated herein by reference.

### BACKGROUND OF THE INVENTION

1. Field of the Invention

The present invention relates generally to the design and use of medical devices, and more particularly to the design and use of an implantable port for establishing temporary access to a patient's vascular system for hemodialysis and other extracorporeal blood treatments, injections, and infusions.

Access to a patient's vascular system can be established by a variety of temporary and permanently implanted devices. Most simply, temporary access can be provided by the direct percutaneous introduction of a needle through the patient's skin and into a blood vessel. While such a direct approach is relatively simple and suitable for applications, such as intravenous feeding, intravenous drug delivery, and other applications which are limited in time, they are not suitable for hemodialysis and other extracorporeal procedures that must be repeated periodically, often for the lifetime of the patient.

For hemodialysis and other extracorporeal treatment regimens, a variety of implantable ports have been proposed over the years. Typically, the port includes a chamber and an access region, such as a septum, where the chamber is attached to an implanted catheter which in turn is secured to a blood vessel. In the case of veins, the catheter is typically indwelling and in the case of arteries, the catheter may be attached by conventional anastomosis.

Of particular interest to the present invention, implantable ports typically include a needle-penetrable septum which permits the percutaneous penetration of a needle into the internal chamber. The chamber, in turn, is connected to one end of the catheter, and the other end of the catheter is indwelling in the blood vessel. While workable, such designs suffer from a number of problems. Repeated penetration of the septum often leads to degradation over time, presenting a substantial risk of small particulates entering the blood stream and/or need to periodically replace the port. Second, the passage of blood through the chamber or plenum will often encounter regions of turbulence or low flow, either of which can degrade the quality of blood over time, thus limiting the flow rates that can be achieved which is a particular problem in hemodialysis access.

For these reasons, it would be desirable to provide improved implantable access ports for percutaneously accessing a patient's blood vessels, including both arteries and veins. The access ports will have septums designed to reduce degradation during use and will be configured to provide relatively high flow rates with minimum blood degradation. The ports will further be configured to facilitate replacement of the port and/or the septum without the need to remove the implanted catheter. Ports according to the present invention will meet at least some of these objectives.

2. Description of the Background Art

U.S. Pat. No. 7,803,143 (Tallarida et al.) shows an implantable hemodialysis access device with multiple ports allowing sequential needle access. U.S. Pat. No. 7,850,666 (Schon et al.) shows a rectangular hemodialysis access port that can have support struts extending across a chamber 120. U.S. Pat. No. 4,857,053 (Dalton) shows an implantable drug delivery device with a matrix septum material for accommodating multiple needle punctures. US Patent Publ. 20120184925 (Grant) shows a multiple septum port. US Patent Publ. 20090118683 (Hanson et al.) shows a dual reservoir implantable access port. U.S. Pat. No. 8,608,713 (Beasley et al.) shows an implantable access device with multiple ports. U.S. Pat. No. 8,079,990 (Powley et al.) shows a multi-port vascular access device with a dual lumen catheter. U.S. Pat. No. 5,833,654 (Powers et al.) shows an implantable dual reservoir access port. U.S. Pat. No. 4,692,146 (Hilger) shows another dual access port.

### BRIEF SUMMARY OF THE INVENTION

This present invention provides an improved implantable vascular access port that can be configured to both accommodate a patient's anatomy and have replaceable interchangeable parts without having to remove and replace the entire port assembly. The septum provides windows configured to be palpably distinguishable through a patient's skin after the port has been subcutaneously implanted whereby a user can select a particular one of the at least three access windows for needle penetration by palpating the patient's skin. The windows can be defined by raised ridges or raised pillows, thus allowing the treatment personnel to sequentially select different windows for success access penetrations to reduce patient trauma and the lessen wear of the septum itself. Such structure markedly improves the options for needle access location sites.

These port designs are particularly useful for patients who receive frequent repeated needle punctures, as often as daily or even multiple times in a day. As an example, dialysis patients on average receive a minimum of six needle punctures for hemodialysis access per week. Another example would be immunocompromised patients needing multiple repeated needle access for chemotherapy. Hemodialysis and chemotherapy patients often also receive multiple needle sticks to draw blood to determine the adequacy of their treatments between their usual dialysis and chemotherapy treatments. Enlarging the septum area and making the septum windows more palpable beneath the skin, allows both multiple sites and more precise needle placement. Most importantly, this configuration allows the patients skin, previous needle puncture site, time to heal before the next traumatic needle access. In the prior patented vascular port septum configurations, needle access is in the same spot; repeatedly traumatizing the same overlying skin spot, not allowing proper time to heal. Unfortunately, this same skin spot receiving these repeated needle punctures not having adequate time to heal begins to thin and break down; exposing the implanted port and causing infection in typically medically compromised patients.

A preferred six window port design according to the present invention allows for multiple needle access points to be distributed along the skin in a different window location evenly between the six windows. This would allow sufficient time for the adjacent overlying subcutaneous skin to heal fully before the next needle access. This invention also allows a patient the option of blood dialysis without the need of standard surgery to create vascular fistulas, further allowing very ill patients who are not surgical candidates to receive blood dialysis and medical treatment via this port catheter insertion.

In a first aspect of the present invention, an implantable needle-access port comprises a housing having at least one elongated interior chamber, at least one elongated needle opening along a surface of the housing, and a catheter connector at one end of the interior chamber. A needle-penetrable septum is disposed over the elongated needle opening, wherein the septum includes at least three axially aligned needle access windows wherein the windows are configured to be palpably distinguishable through a patient's skin after the port has been subcutaneously implanted whereby a user can select a particular one of the at least three access windows for needle penetration by palpating the patient's skin.

The housing preferably consists essentially of a rectangular enclosure, typically formed of a metal or hardened plastic, having two major side surfaces and two end surfaces. The at least one elongated needle opening occupies substantially entirely one of the major side surfaces and the catheter connector is on one side of the end surfaces. Lateral struts may be formed to span the needle opening at locations which align with the regions between adjacent windows in the septum when the septum is in place in the needle opening.

In some embodiments, the needle-penetrable septum consists of a single, continuous structure with the windows formed integrally therein. The needle-penetrable septum is typically composed of a self-sealing polymer which is substantially free from metal reinforcement, and the windows are typically formed as well-like receptacles or pillow-like projections having substantially equal dimensions which occupy the entire area of the needle opening. Forming these features may be accomplished by any conventional polymer process, such as molding, laminating, casting, or the like.

In other embodiments, the needle-penetrable septum is removable and replaceable in the needle opening after implantation, and the ports may further include a vascular access catheter which is attachable to the catheter connector on the housing.

In still other embodiments, the vascular access catheter is removably attached to the catheter connector and at least one of the housing and the catheter is configured to be replaced after implantation.

In a second aspect of the present invention, an implantable hemodialysis port comprises a housing having an elongated interior inlet chamber and an elongated interior outlet chamber. Each chamber has an elongated needle opening disposed along an upper surface of the housing and a catheter connector at one end of the chamber. A needle-penetrable septum is disposed over the elongated needle opening of each chamber, and each septum includes at least three axially aligned access windows wherein the windows are configured to be palpably distinguishable through a patient's skin after the port has been subcutaneously implanted. Thus, a user can select a particular one of the at least three access windows over each chamber for needle penetration by palpating the patient's skin.

The housing optionally consists essentially of a rectangular enclosure, typically being formed from metal or hard plastic, having two major side surfaces and two end surfaces. The elongated inlet chamber and the elongated outlet chamber are arranged side-by-side, and the needle opening for the inlet chamber and the needle opening for the outlet chamber are also arranged side-by-side over the said chambers and occupy substantially entirely one of the major side surfaces. The catheter connector is on one of the end surfaces. Lateral struts may be provided to span each needle opening at locations which align with the regions between adjacent windows in the septum when the septum is in place in the needle opening.

Each needle-penetrable septum may consist of a single, continuous structure with the windows formed integrally therein. Alternatively, the two needle-penetrable septums together may consist of a single, continuous structure with the windows formed integrally therein. In both cases, the needle-penetrable septum is preferably composed of a self-sealing polymer which is substantially free from metal reinforcement. The windows in each septum are usually formed as well-like receptacles or pillow-like projections having substantially equal dimensions which occupy substantially the entire area of the respective needle opening. Forming these features may be accomplished by any conventional polymer process, such as molding, laminating, casting, or the like.

Each needle-penetrable septum may be removable and replaceable in the respective needle opening after implantation. The ports may further comprise a vascular access catheter which is attached to each catheter connector on the housing, and each vascular access catheter may be removably attached to the catheter connector and at least one of the housing and the catheter is configured to be replaced after implantation.

In a third aspect of the present invention, a method for performing dialysis comprises palpating a patient's skin over a previously implanted port as described above. As a result of said palpitating, a user can identify (a) one window in the needle-penetrable septum over the inlet chamber and (b) one window in the needle-penetrable septum over the outlet chamber. An un-dialyzed blood needle is penetrated into the identified one window in the septum over the inlet chamber for aspiration, and a blood outlet needle is penetrated into the identified one window in the septum over the outlet chamber. The other non-identified windows in each of the inlet and outlet septums remain available for subsequent needle penetration. Blood drawn through the blood inlet needle is dialyzed and dialyzed blood is returned through the blood return outlet needle.

### BRIEF DESCRIPTION OF THE DRAWINGS

The associated drawings illustrate the invention:

FIG. 1 is an exterior oblique perspective view of an implantable high volume flow rate dual lumen vascular access port constructed in accordance with the principles of the present invention.

FIG. 1A is an exterior oblique perspective view of an alternative embodiment of an implantable high volume flow rate dual lumen vascular access port constructed in accordance with the principles of the present invention.

FIG. 2 is an exterior anterior top view of the port illustrated in FIG. 1.

FIG. 3 is a lateral side view of the port illustrated in FIG. 1

FIG. 4 is a view of the non-catheter end of the port illustrated in FIG. 1.

FIG. 5 is a view of the catheter attachment end of the port illustrated in FIG. 1.

FIG. 6 is a bottom view of the port illustrated in FIG. 1.

FIG. 7 is a sectional view taken along line 7-7 in FIG. 2.

FIG. 8 is a sectional view taken along line 8-8 in FIG. 3.

FIG. 9 is a sectional view taken along line 9-9 in FIG. 2.

FIG. 10 illustrates placement of a port and catheter assembly in a patient's right internal jugular vein for performing hemodialysis in accordance with the principles of the present invention.

FIG. 11 illustrates a further alternative port including an additional access septum and separate passage for drug infusion or other purposes.

FIG. 12 is a sectional view of a catheter for connecting to the port of FIG. 11 and having a third lumen for connecting to the additional septum.

### DETAILED DESCRIPTION OF THE INVENTION

This invention is a subcutaneous implantable dual lumen high flow volume vascular port catheter, designed to rapidly both deliver fluid into and withdraw fluid from the patient's bloodstream. Access into the solid based reservoir dual lumens is via a non-coring needle through an anterior non-coring needle penetrable self-sealing septum. This septum is self-sealing, allowing for multiple repeated non-coring needle placements without leaking from the septum. The septum holds and seals the non-coring needle in place during fluid movement. The septum is designed for subcutaneous tactile palpation for localization of non-coring needle placement. The septum has a trough rectangular shape with six windows in which to access the two lumens.

Within the base, each of the lumens has a large diameter caliber outlet stem. The stem connects to a high pressure catheter that is placed into the patient's bloodstream. Flushing positive pressure through the needle into the reservoir would allow fluids to be sent into the patient's blood stream. Negative pressure aspiration through the needle within the reservoir will allow blood to be removed from the blood stream.

The calibers of the outlet connections and of the high pressure catheter are specifically designed to allow large volume high flow rates to accommodate for functions such as but not limited to dialysis. The port assembly is designed with interchangeable replaceable parts for possible maintenance repairs.

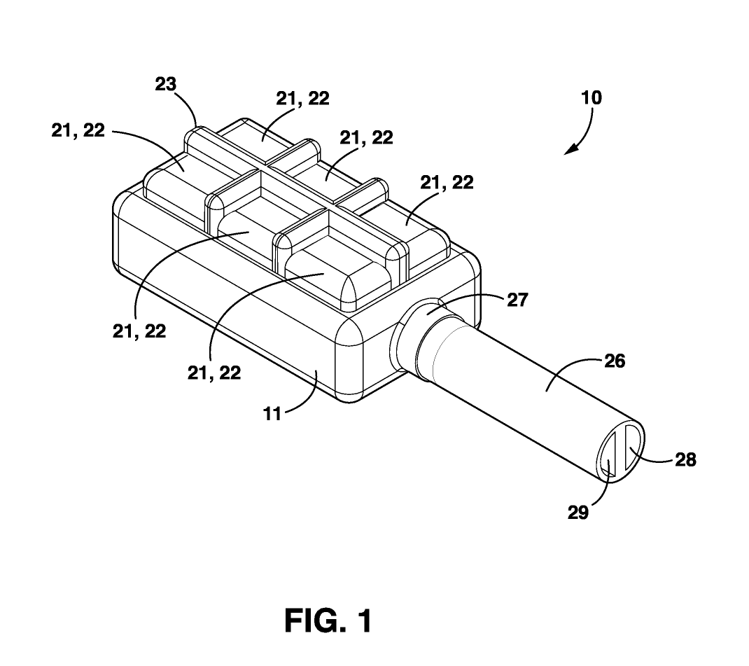

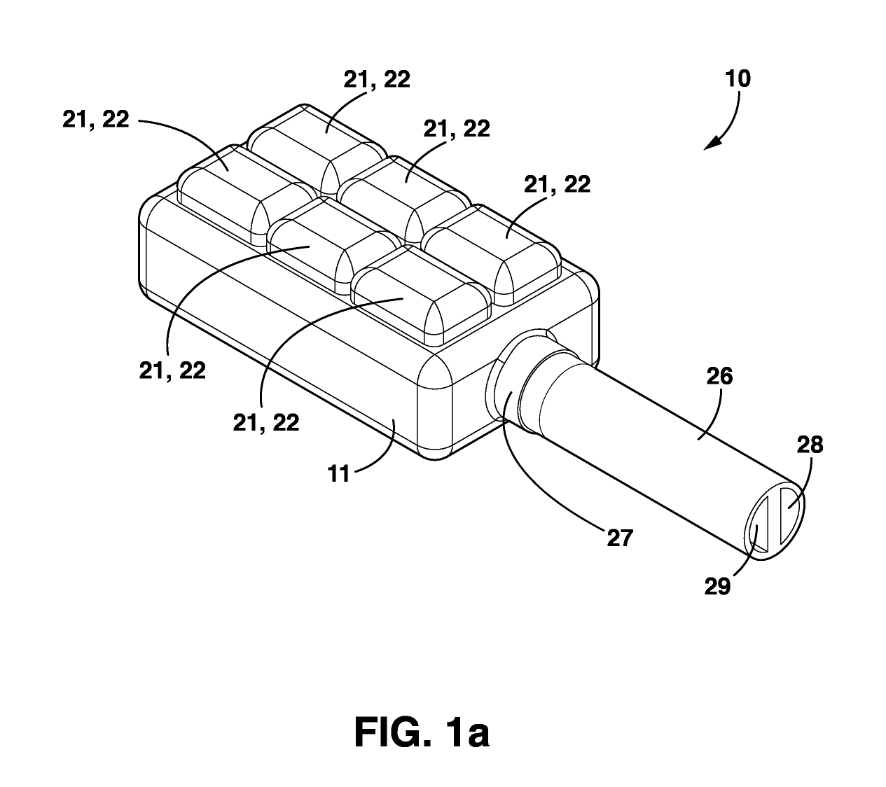

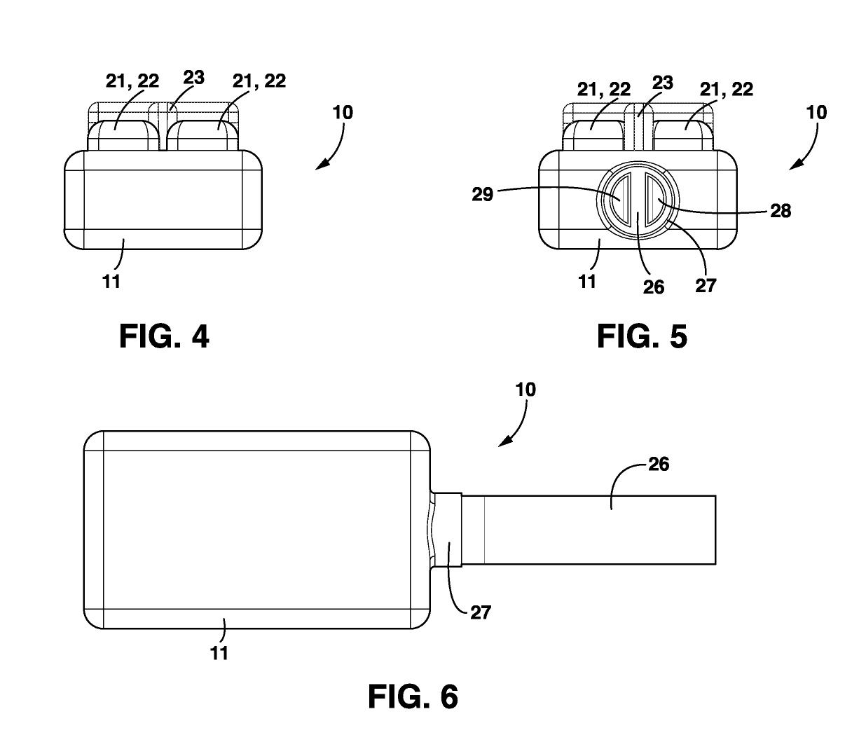

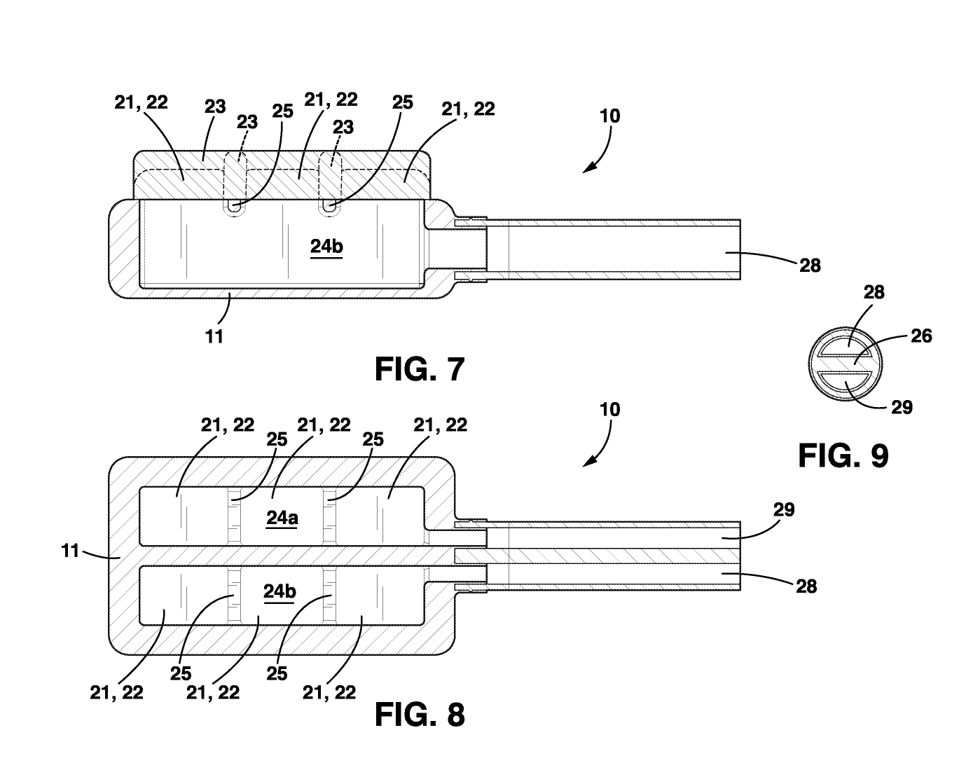

Referring now to FIGS. 1-9, an implantable high volume flow rate dual lumen vascular access port (10) comprises a needle impenetrable housing (11) having an openings 21 on an upper surface thereof that receive one or more septum structure(s) that includes six septum window wells 22 for passing non-coring needles to access the chambers 24a and 24b, as best seen in FIGS. 7 and 8. The individual wells 22 may be defined by raised ridges (23) of the septum (typically molded or otherwise formed into the septum) allowing for tactile palpation of the septum window wells when beneath the skin. A removable catheter (26) is fastened to the housing (11) via a locking ring (27).

FIG. 1A illustrates an alternative embodiment (10′) of the port where the windows are formed as pillows (22′) separated by troughs or channels 23′ rather than as wells.

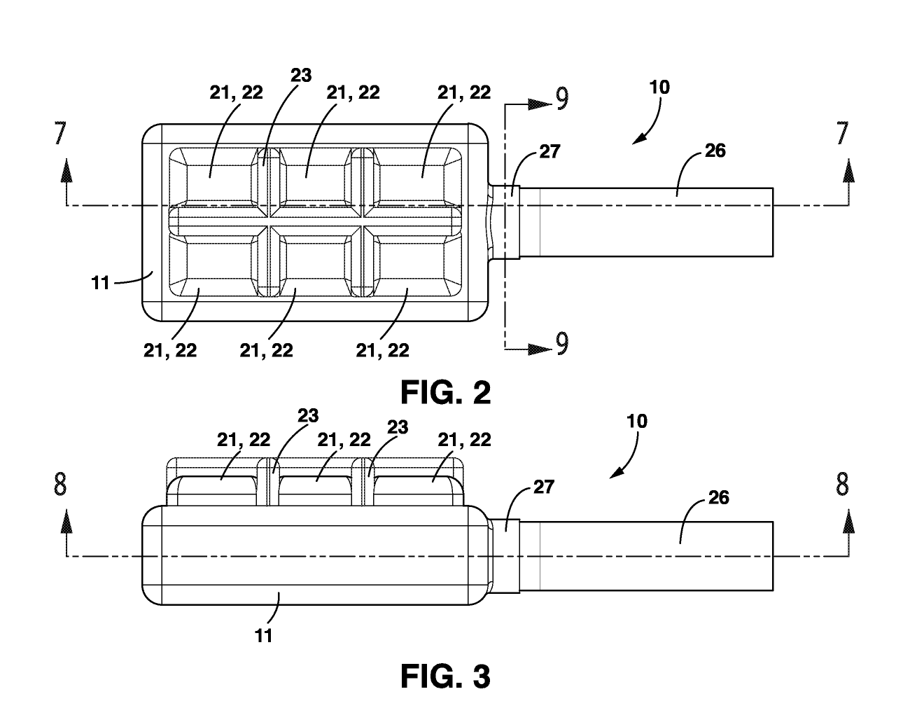

Once implanted subcutaneously, an anterior or top side of the port as seen in FIG. 2 will abut the subcutaneous skin for tactile palpation of the septum windows (22) and septum ridges (23).

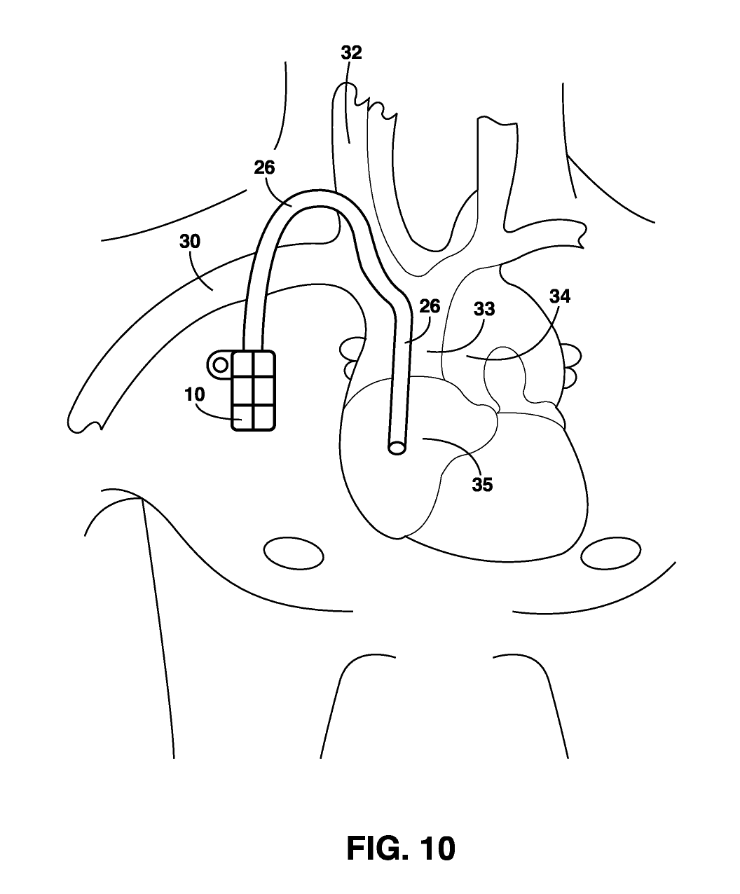

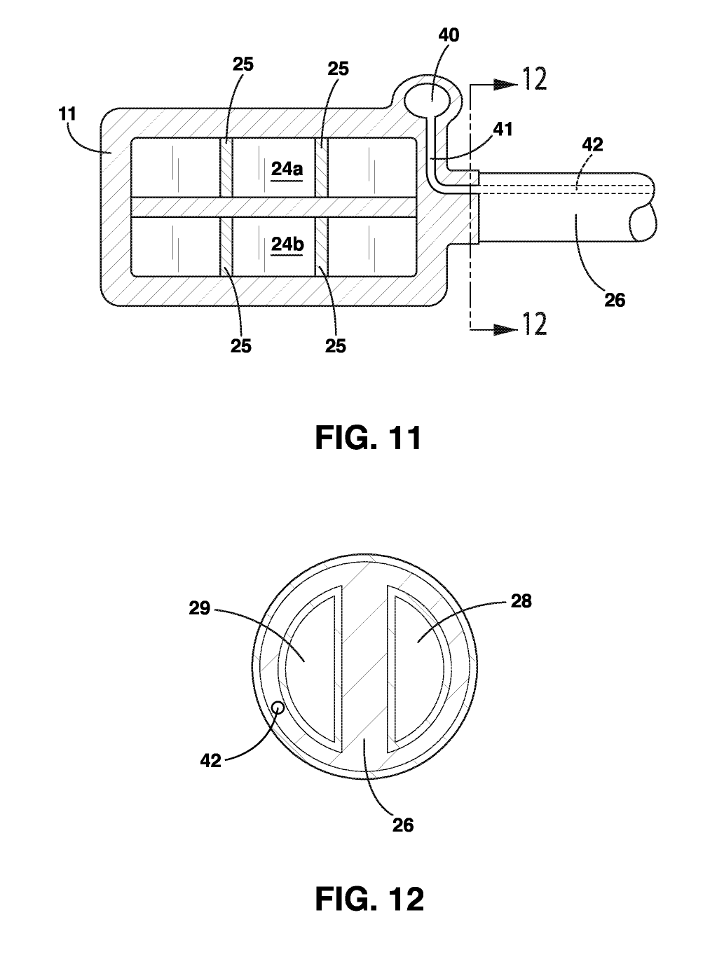

As seen in FIGS. 5 and 9, the catheter (26) has two lumens (28) and (29) which are in direct communication with the chambers (24a) and (24b) illustrated in FIGS. 7 and 8.

FIG. 7 is a cut side view of (10) illustrated in FIG. 2. Each chamber (24a) and (24b) is in open direct communication with the associated catheter lumen (28 and (29), respectively). As shown in FIG. 8, solid struts (25) may be provided to support the overlying septum or septums. There may be two separate septums, each including three windows, with one septum positioned over an opening at the top of each camber (24a) and (24b). Alternatively, there may be a single septum having six windows. In either case, the septum will be contiguous and attached to the housing (11) to provide a fluid-tight seal.

The chambers (24a) and (24b) are in open direct communication with the lumens (28) and (29) in catheter (26) This allows fluid such as blood to be directly infused into circulation or aspirated from circulation via the chambers and through the catheter (26).

As shown in FIG. 10, the port catheter (10) illustrated in FIG. 1 is placed subcutaneously in a patient's vascular anatomy with the catheter of (26) inserted into the right internal jugular vein (32) and with the tip of the catheter (26) in the right atrium (35). The right subclavian vein (30) the superior vena cava (33) , and the aorta(34) are also illustrated.

As seen in FIGS. 12 and 13, the catheter (26) has three lumens (42), (28) and (29) which are in direct communication with the chambers (40), (24a) and (24b) previously illustrated in FIGS. 7 and 8.

What is claimed is:

1. An implantable needle-access port comprising:
a housing having at least one elongated interior chamber, at least one elongated needle opening along a surface of the housing, and a catheter connector at one end of the interior chamber; and
a needle-penetrable septum disposed over the elongated needle opening, wherein the septum includes at least three axially aligned needle access windows wherein the windows are configured to be palpably distinguishable through a patient's skin after the port has been subcutaneously implanted whereby a user can select a particular one of the at least three access windows for needle penetration by palpating the patient's skin.

2. An implantable port as in claim 1, wherein the housing consists essentially of a rectangular enclosure having two major side surfaces and two end surfaces, wherein the at least one elongated needle opening occupies substantially entirely one of the major side surfaces and the catheter connector is on one side of the end surfaces.

3. An implantable port as in claim 2, wherein the housing has lateral struts spanning the needle opening at locations which align with the regions between adjacent windows in the septum when the septum is in place in the needle opening.

4. An implantable port as in claim 1, wherein the needle-penetrable septum consists of a single, continuous structure with the windows formed integrally therein.

5. An implantable port as in claim 4, wherein the needle-penetrable septum is composed of a self-sealing polymer which is substantially free from metal reinforcement.

6. An implantable port as in claim 5, wherein the windows are formed as well-like receptacles or pillow-like projections having substantially equal dimensions which occupy the entire area of the needle opening.

7. An implantable port as in claim 1, wherein the needle-penetrable septum is removable and replaceable in the needle opening after implantation.

8. An implantable port as in claim 7, further comprising a vascular access catheter which is attachable to the catheter connector on the housing.

9. An implantable port as in claim 8, wherein the vascular access catheter is removably attached to the catheter connector and at least one of the housing and the catheter is configured to be replaced after implantation.

10. An implantable hemodialysis port comprising:
a housing having an elongated interior inlet chamber and an elongated interior outlet chamber, wherein each chamber has an elongated needle opening disposed along an upper surface of the housing and a catheter connector at one end of the chamber;
and a needle-penetrable septum disposed over the elongated needle opening of each chamber, wherein each septum includes at least three axially aligned access windows wherein the windows are configured to be palpably distinguishable through a patient's skin after the port has been subcutaneously implanted whereby a user can select a particular one of the at least three access windows for needle penetration by palpating the patient's skin.

11. An implantable port as in claim 10, wherein the housing consists essentially of a rectangular metal enclosure having two major side surfaces and two end surfaces, wherein the elongated inlet chamber and the elongated outlet chamber are arranged side-by-side and the needle opening for the inlet chamber and the needle opening for the outlet chamber are arranged side-by-side over the said chambers and occupy substantially entirely one of the major side surfaces and the catheter connector is on one of the end surfaces.

12. An implantable port as in claim 11, having lateral struts spanning each needle opening at locations which align with the regions between adjacent windows in the septum when the septum is in place in the needle opening.

13. An implantable port as in claim 10, wherein each needle-penetrable septum consists of a single, continuous structure with the windows formed integrally therein.

14. An implantable port as in claim 10, wherein the two needle-penetrable septums together consist of a single, continuous structure with the windows formed integrally therein.

15. An implantable port as in claim 13, wherein each needle-penetrable septum is composed of a self-sealing polymer which is substantially free from metal reinforcement.

16. An implantable port as in claim 15, wherein the windows in each septum are formed as well-like receptacles or pillow-like projections having substantially equal dimensions which occupy substantially the entire area of the respective needle opening.

17. An implantable port as in claim 10, wherein each needle-penetrable septum is removable and replaceable in the respective needle opening after implantation.

18. An implantable port as in claim 17, further comprising a vascular access catheter which is attached to each catheter connector on the housing.

19. An implantable port as in claim 18, wherein each vascular access catheter is removably attached to the catheter connector and at least one of the housing and the catheter is configured to be replaced after implantation.

20. A method for performing dialysis, said method comprising:
palpating a patient's skin over a previously implanted port according to claim 10;
identifying as a result of said palpitating (a) one window in the needle-penetrable septum over the inlet chamber and (b) one window in the needle-penetrable septum over the outlet chamber; penetrating a un-dialyzed blood needle into the identified one window in the septum over the inlet chamber for aspiration; and
penetrating a blood outlet needle into the identified one window in the septum over the outlet chamber; and
wherein the other non-identified windows in each of the inlet and outlet septums remain available for subsequent needle penetration; and
dialyzing blood drawn through the blood inlet needle and returning dialyzed blood through the blood return outlet needle.
